(19) United States
(12) Patent Application Publication
(10) Pub. No.: US 2022/0192541 A1
(43) Pub. Date: Jun. 23, 2022

(54) HEARING ASSESSMENT USING A HEARING INSTRUMENT

(71) Applicant: Starkey Laboratories, Inc., Eden Prairie, MN (US)

(72) Inventors: Christine Marie Tan, Eden Prairie, MN (US); Kevin Douglas Seitz-Paquette, Minneapolis, MN (US)

(21) Appl. No.: 17/603,431

(22) PCT Filed: Apr. 17, 2020
(86) PCT No.: PCT/US2020/028772

§ 371 Date: Oct. 13, 2021

Related U.S. Application Data
(60) Provisional application No. 62/835,664, filed on Apr. 18, 2019.

(51) Int. Cl. A61B 5/12; H04R 25/00; A61B 5/11

(52) U.S. Cl. CPC A61B 5/125; H04R 25/405; A61B 2503/04; H04R 25/30; A61B 5/1114; H04R 25/407

(57) ABSTRACT

A computing system includes a memory and at least one processor. The memory is configured to store motion data indicative of motion of a hearing instrument. The at least one processor is configured to determine, based on the motion data, whether a user of the hearing instrument perceived a sound. The at least one processor is further configured to output data indicating whether the user perceived the sound.

This patent application claims the benefit of U.S. Provisional Patent Application No. 62/835,664, filed Apr. 18, 2019, the entire content of which is incorporated by reference.

### TECHNICAL FIELD

This disclosure relates to hearing instruments.

### BACKGROUND

A hearing instrument is a device designed to be worn on, in, or near one or more of a user's ears. Example types of hearing instruments include hearing aids, earphones, earbuds, telephone earpieces, cochlear implants, and other types of devices. In some examples, a hearing instrument may be implanted or osseointegrated into a user. It may be difficult to tell whether a person is able to hear a sound. For example, infants and toddlers may be unable to reliably provide feedback (e.g., verbal acknowledgment, a button press) to indicate whether they can hear a sound.

### SUMMARY

In general, this disclosure describes techniques for monitoring a person's hearing ability and performing hearing assessments using hearing instruments. A computing device may determine whether a user of a hearing instrument has perceived a sound based at least in part on motion data generated by the hearing instrument. For instance, the user may turn his or her head towards a sound and a motion sensing device (e.g., an accelerometer) of the hearing instrument may generate motion data indicating the user turned his or her head. The computing device may determine that the user perceived the sound if the user turns his or her head within a predetermined amount of time of the sound occurring. In this way, the computing device may more accurately determine whether the user perceived the sound, which may enable a hearing treatment provider (e.g., an audiologist or hearing instrument specialist) or other type of person to better monitor, diagnose and/or treat the user for hearing impairments.

In one example, a computing system includes a memory and at least one processor. The memory is configured to store motion data indicative of motion of a hearing instrument. The at least one processor is configured to determine, based on the motion data, whether a user of the hearing instrument perceived a sound, and responsive to determining whether the user perceived the sound, output data indicating whether the user perceived the sound.

In another example, a method is described that includes receiving, by at least one processor, motion data indicative of motion of a hearing instrument; determining, by the at least one processor, based on the motion data, whether a user of the hearing instrument perceived a sound; and responsive to determining whether the user perceived the sound, outputting, by the one or more processors, data indicating whether the user perceived the sound.

In another example, a computer-readable storage medium is described. The computer-readable storage medium includes instructions that, when executed by at least one processor of a computing device, cause at least one processor to: receive motion data indicative of motion of a hearing instrument; determine, based on the motion data, whether a user of the hearing instrument perceived a sound; and responsive to determining whether the user perceived the sound, output data indicating whether the user perceived the sound.

In yet another example, the disclosure describes means for receiving motion data indicative of motion of a hearing instrument; determining whether a user of the hearing instrument perceived a sound; and responsive to determining whether the user perceived the sound, outputting data indicating whether the user perceived the sound.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 is a conceptual diagram illustrating an example system for performing hearing assessments, in accordance with one or more aspects of the present disclosure.

FIG. 2 is a block diagram illustrating an example of a hearing instrument, in accordance with one or more aspects of the present disclosure.

FIG. 3 is a conceptual diagram illustrating an example computing system, in accordance with one or more aspects of the present disclosure.

FIG. 4 illustrates graphs of example motion data, in accordance with one or more aspects of the present disclosure.

FIG. 5 is a flow diagram illustrating example operations of a computing device, in accordance with one or more aspects of the present disclosure.

### DETAILED DESCRIPTION

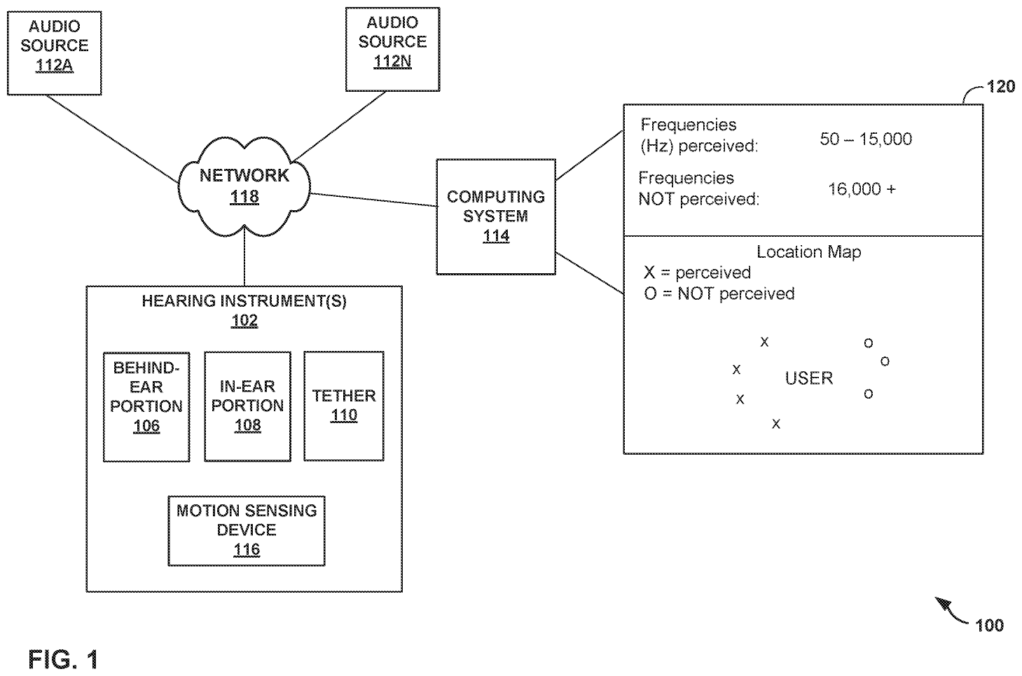

FIG. 1 is a conceptual diagram illustrating an example system for performing hearing assessments, in accordance with one or more aspects of the present disclosure. System 100 includes at least one hearing instrument 102, one or more audio sources 112A-N (collectively, audio sources 112), a computing system 114, and communication network 118. System 100 may include additional or fewer components than those shown in FIG. 1.

Hearing instrument 102, computing system 114, and audio sources 112 may communicate with one another via communication network 118. Communication network 118 may comprise one or more wired or wireless communication networks, such as cellular data networks, WIFI™ networks, BLUETOOTH™ networks, the Internet, and so on.

Hearing instrument 102 is configured to cause auditory stimulation of a user. For example, hearing instrument 102 may be configured to output sound. As another example, hearing instrument 102 may stimulate a cochlear nerve of a user. As the term is used herein, a hearing instrument may refer to a hearing instrument that is used as a hearing aid, a personal sound amplification product (PSAP), a headphone set, a bearable, a wired or wireless earbud, a cochlear implant system (which may include cochlear implant magnets, cochlear implant transducers, and cochlear implant processors), or another type of device that provides auditory stimulation to a user. In some instances, hearing instruments 102 may be worn. For instance, a single hearing instrument 102 may be worn by a user (e.g., with unilateral hearing loss). In another instance, two hearing instruments, such as hearing instrument 102, may be worn by the user (e.g., with bilateral hearing loss) with one instrument in each ear. In some examples, hearing instruments 102 are implanted on the user (e.g, a cochlear implant that is implanted within the ear canal of the user). The described techniques are applicable to any hearing instruments that provide auditory stimulation to a user.

In some examples, hearing instrument 102 is a hearing assistance device. In general, there are three types of hearing assistance devices. A first type of hearing assistance device includes a housing or shell that is designed to be worn in the ear for both aesthetic and functional reasons. The housing or shell encloses electronic components of the hearing instrument. Such devices may be referred to as in-the-ear (ITE), in-the-canal (ITC), completely-in-the-canal (CIC), or invisible-in-the-canal (IIC) hearing instruments.

A second type of hearing assistance device, referred to as a behind-the-ear (BTE) hearing instrument, includes a housing worn behind the ear which may contain all of the electronic components of the hearing instrument, including the receiver (i.e., the speaker). An audio tube conducts sound from the receiver into the user's ear canal.

A third type of hearing assistance device, referred to as a receiver-in-canal (RIC) hearing instrument, has a housing worn behind the ear that contains some electronic components and further has a housing worn in the ear canal that contains some other electronic components, for example, the receiver. The behind the ear housing of a RIC hearing instrument is connected (e.g., via a tether or wired link) to the housing with the receiver that is worn in the ear canal. Hearing instrument 102 may be an ITE, ITC, CIC, IIC, BTE, RIC, or other type of hearing instrument.

In the example of FIG. 1, hearing instrument 102 is configured as a RIC hearing instrument and includes its electronic components distributed across three main portions: behind-ear portion 106, in-ear portion 108, and tether 110. In operation, behind-ear portion 106, in-ear portion 108, and tether 110 are physically and operatively coupled together to provide sound to a user for hearing. Behind-ear portion 106 and in-ear portion 108 may each be contained within a respective housing or shell. The housing or shell of behind-ear portion 106 allows a user to place behind-ear portion 106 behind his or her ear whereas the housing or shell of in-ear portion 108 is shaped to allow a user to insert in-ear portion 108 within his or her ear canal.

In-ear portion 108 may be configured to amplify sound and output the amplified sound via an internal speaker (also referred to as a receiver) to a user's ear. That is, in-ear portion 108 may receive sound waves (e.g., sound) from the environment and converts the sound into an input signal. In-ear portion 108 may amplify the input signal using a pre-amplifier, may sample the input signal, and may digitize the input signal using an analog-to-digital (A/D) converter to generate a digitized input signal. Audio signal processing circuitry of in-ear portion 108 may process the digitized input signal into an output signal (e.g., in a manner that compensates for a user's hearing deficit). In-ear portion 108 then drives an internal speaker to convert the output signal into an audible output (e.g. sound waves).

Behind-ear portion 106 of hearing instrument 102 is configured to contain rechargeable or non-rechargeable power source that provides electrical power, via tether 110, to in-ear portion 108. In some examples, in-ear portion 108 includes its own power source, and behind-ear portion 106 supplements the power source of in-ear portion 108.

Behind-ear portion 106 may include various other components, in addition to a rechargeable or non-rechargeable power source. For example, behind-ear portion 106 may include a radio or other communication unit to serve as a communication link or communication gateway between hearing instrument 102 and the outside world. Such a radio may be a multi-mode radio, or a software-defined radio configured to communicate via various communication protocols. In some examples, behind-ear portion 106 includes a processor and memory. For example, the processor of behind-ear portion 106 may be configured to receive sensor data from sensors within in-ear portion 108 and analyze the sensor data or output the sensor data to another device (e.g., computing system 114, such as a mobile phone). In addition to sometimes serving as a communication gateway, behind-ear portion 106 may perform various other advanced functions on behalf of hearing instrument 102; such other functions are described below with respect to the additional figures.

Tether 110 forms one or more electrical links that operatively and communicatively couple behind-ear portion 106 to in-ear portion 108. Tether 110 may be configured to wrap from behind-ear portion 106 (e.g., when behind-ear portion 106 is positioned behind a user's ear) above, below, or around a user's ear, to in-ear portion 108 (e.g., when in-ear portion 108 is located inside the user's ear canal). When physically coupled to in-ear portion 108 and behind-ear portion 106, tether 110 is configured to transmit electrical power from behind-ear portion 106 to in-ear portion 108. Tether 110 is further configured to exchange data between portions 106 and 108, for example, via one or more sets of electrical wires.

Hearing instrument 102 may detect sound generated by one or more audio sources 112 and may amplify portions of the sound to assist the user of hearing instrument 102 in hearing the sound. Audio sources 112 may include animate or inanimate objects. Inanimate objects may include an electronic device, such as a speaker. Inanimate objects may include any object in the environment, such as a musical instrument, a household appliance (e.g., a television, a vacuum, a dishwasher, among others), a vehicle, or any other object that generates sound waves (e.g., sound). Examples of animate objects include humans and animals, robots, among others. In some examples, hearing instrument 102 may include one or more of audio sources 112. In other words, the receiver or speaker of hearing instrument 102 may be an audio source that generates sound.

Audio sources 112 may generate sound in response to receiving a command from computing system 114. The command may include a digital representation of a. sound. For example, a hearing treatment provider (e.g., an audiologist or hearing instrument specialist) may operate computing system 114 and may provide a user input (e.g., a touch input, a mouse input, a keyboard input, among others) to computing system 114 to send a command to audio sources 112 to generate sound. For example, audio source 112A may include an electronic device that includes a speaker and may generate sound in response to receiving the digital representation of the sound from computing system 114. Examples of computing system 114 include a mobile phone (e.g., a smart phone), a wearable computing device (e.g., a smart watch), a laptop computing, a desktop computing device, a television, a distributed computing system (e.g., a “cloud” computing system), or any type of computing system.

In some instances, audio sources 112 generate sound without receiving a command from computing system 114. In one instance, audio source 112N may be a human that generates sound via speaking, clapping, or performing some other action. For instance, audio source 112N may include a parent that generates sound by speaking to a child (e.g., calling the name of the child). A user of hearing instrument 102 may turn his or her head in response to hearing sound generated by one or more of audio sources 112.

In some examples, hearing instrument 102 includes at least one motion sensing device 116 configured to detect motion of the user (e.g., motion of the user's head). Hearing instrument 102 may include a motion sensing device disposed within behind-ear portion 106, within in-ear portion 108, or both. Examples of motion sensing devices include an accelerometer, a gyroscope, a magnetometer, among others. Motion sensing device 116 generates motion data indicative of the motion. For instance, the motion data may include unprocessed data and/or processed data representing the motion. Unprocessed data may include acceleration data indicating an amount of acceleration in one or more dimensions (e.g., x, y, and/or z-dimensions) over time or gyroscope data indicating a speed or rate of rotation in one or more dimensions over time. In some examples, the motion data may include processed data, such as summary data indicative of the motion. For instance, in one example, the summary data may include data indicating a degree of head rotation (e.g., degree of pitch, yaw, and/or roll) of the user's head. In some instances, the motion data indicates a time associated with the motion, such as a timestamp indicating a time at which the user turned his or her head or a plurality of timestamps indicating a respective time at which various portions of unprocessed data was received.

Computing system 114 may receive sound data associated with one or more sounds generated by audio sources 112 in some examples, the sound data includes a timestamp that indicates a time associated with a sound generated by audio sources 112. In one example, computing system 114 instructs audio sources 112 to generate the sound such that the time associated with the sound is a time at which computing system 114 instructed audio sources 112 to generate the sound or a time at which the sound was generated by audio sources 112. In one scenario, hearing instrument 102 and/or computing system 114 may detect sound occurring in the environment that is not caused by computing system 114 (e.g., naturally-occurring sounds rather than sounds generated by an electronic device, such as a speaker). In such scenarios, the time associated with the sound generated by audio sources 112 is a time at which the sound was detected (e.g., by hearing instrument 102 and/or computing system 114). In some examples, the sound data may include the data indicating the time associated with the sound, data indicating one or more characteristics of the sound (e.g., intensity, frequency, etc.), a transcript of the sound (e.g., when the sound includes human or computer-generated speech), or a combination thereof. In one example, the transcript of the sound may indicate one or more keywords included in the sound (e.g., the name of a child wearing hearing instrument 102).

In accordance with techniques of this disclosure, computing system 114 may perform a diagnostic assessment of the user's hearing (also referred to as a hearing assessment). Computing system 114 may perform a hearing assessment in a supervised setting (e.g., in a clinical setting monitored by a hearing treatment provider). In another example, computing system 114 performs a hearing assessment in an unsupervised setting. For example, computing system 114 may perform an unsupervised hearing assessment if a patient is unable or unwilling to cooperate with a supervised hearing assessment.

Computing system 114 may perform the hearing assessment to determine whether the user perceives a sound. Computing system 114 may determine whether the user perceived the sound based at least in part on the motion data and the sound data. In one example, computing system 114 determines whether the user perceived the sound based on determining whether a degree of motion of the user satisfies a motion threshold and whether an amount of time between the time associated with the sound and the time associated with the motion satisfies a time threshold.

Computing system 114 may determine whether a degree of motion of the user satisfies a motion threshold. In some examples, computing system 114 determines the degree of rotation based on the motion data. In one example, computing system 114 may determine an initial or reference head position (e.g., looking straight forward) at a first time, determine a subsequent head position of the user at a second time based on the motion data, and determine a degree of rotation between the initial head position and the subsequent head position. For example, computing system 114 may determine the degree of rotation includes an approximately 45-degree rotation (e.g., about an axis defined by the user's spine). Computing system 114 may compare the degree of rotation to a motion threshold to determine whether the user perceived the sound.

In some instances, computing system 114 determines the motion threshold. For instance, computing system 114 may determine the motion threshold based on one or more characteristics of the user (e.g., age, attention span, cognition, motor function, etc.), one or more characteristics of the sound (e.g., frequency, intensity, etc.), or both. In one instance, computing system 114 may assign a relatively high motion threshold when the user is one age (e.g., six months) and a relatively low motion threshold when the user is another age (e.g., three years). For instance, a child under a certain age may have insufficient muscle control to rotate his or her head in small increments, such that the motion threshold for such children may be relatively high compared to older children who are able to rotate their heads in smaller increments (e.g., with more precision). As another example, computing system 114 may assign a relatively high motion threshold to sounds at a certain intensity level and a relatively low motion threshold to sounds at another intensity level. For example, a user may turn his or her head a relatively small amount when perceiving a relatively quiet noise and may turn his or her head a relatively large amount when perceiving a loud noise. As yet another example, computing system 114 may determine the motion threshold based on the direction of the source of the sound. For example, computing system 114 may assign a relatively high motion threshold if the source of the sound is located behind the user and a relatively low motion threshold if the source of the sound is located nearer the front of the user.

Computing system 114 may determine whether an amount of elapsed time between the time associated with the sound and the time associated with the motion satisfies a time threshold. In some examples, computing system 114 determines the time threshold based on one or more characteristics of the user (e.g., age, attention span, cognition, motor function, etc.). For example, computing system 114 may assign a relatively high time threshold when the user is a certain age (e.g., one year) and a relatively low time threshold when the user is another age. For instance, children may respond to sounds faster as they age while elderly users may respond more slowly in advanced age.

Computing system 114 may determine that the user did not perceive the sound in response to determining that the degree of rotation does not satisfy (e.g., is less than) a motion threshold or in response to determining that the amount of elapsed time satisfies (e.g., is greater than or equal to) a time threshold. Computing system 114 may determine that the user perceived the sound in response to determining that the degree of rotation satisfies (e.g., is greater than) a motion threshold and that the amount of elapsed time does not satisfy (e.g., is less than) the time threshold.

Additionally, or alternatively, computing system 114 may determine whether the user perceived the sound based on a direction in which the user turned his or her head. Computing system 114 may determine the motion direction based on the motion data. For example, computing system 114 may determine whether the user turned his or her head left or right. In some examples, computing system 114 determines whether the user perceived the sound based on whether the user turned his or her head in the direction of the audio source 112 that generated the sound.

Computing system 114 may determine a direction of the audio source 112 that generated the sound. In some examples, computing system 114 outputs a command to a particular audio source 112A to generate sound and determines the direction of the audio source 112 relative to the user (and hence hearing instrument 102) or relative to computing system 114. For example, computing system 114 may store or receive location information (also referred to as data) indicating a physical location of audio source 112A, a physical location of the user, and/or a physical location of computing system 114. In some examples, the information indicating a physical location of audio source 112A, the physical location of the user, and the physical location of computing system 114 may include reference coordinates (e.g., GPS coordinates or coordinates within a building/room reference system) or information specifying a spatial relation between the devices. Computing system 114 may determine a direction of audio source 112A relative to the user or computing system 114 based on the location information of audio source 112A and the user or computing system 114, respectively.

Computing system 114 may determine a direction of audio source 112A relative to the user and/or computing system 114 based on one or more characteristics of sound detected by two or more different devices. In some instances, computing system 114 may receive sound data from a first hearing instrument 102 worn on one side of the user's head and sound data from a second hearing instrument 102 worn on the other side of the user's head (or computing system 114). For instance, computing system 114 may determine audio source 112A is located in a first direction (e.g., to the right of the user) if the sound detected by the first hearing instrument 102 is louder than the sound detected by the second hearing instrument 102 and that the audio source 112A is located in a second direction (e.g., to the left of the user) if the sound detected by the second hearing instrument 102 is louder than the sound detected by the first hearing instrument 102.

Responsive to determining the direction of audio source 112A relative to the user and/or computing system 114, computing system 114 may determine the user perceived the sound in response to determining the user moved his or her head in the direction of audio source 112A. Computing system 114 may determine the user did not perceive the sound in response to determining the user moved his or her head in a direction different than the direction of audio source 112A. In other words, in some examples, computing system 114 may determine the audio source 112A is located to the left of the user and that the user turned his head right, such that computing system 114 may determine the user did not perceive the sound (e.g., rather, the user may have coincidentally turned his head to the right at approximately the same time the audio source 112A generated the sound). Said another way, computing system 114 may determine whether the user perceived the sound based on whether the direction of the motion is aligned with the direction of the audio source 112A. For instance, computing system 114 may determine the user perceived the sound in response to determining the direction of motion is aligned with the direction of audio source 112A and may determine the user did not perceive the sound in response to determining the direction of the motion is not aligned with the direction of audio source 112A.

Computing system 114 may output data indicating whether the user perceived the sound. For example, computing system 114 may output a graphical user interface (GUI) 120 indicating characteristics of sounds perceived by the user and sounds not perceived by the user. In some examples, the characteristics of the sounds include intensity, frequency, location of the sound relative to the user, or a combination therein. In the example of FIG. 1, GUI 120 indicates the frequencies of sounds perceived by the user, and the locations from which sounds were received and whether the sounds were perceived. As another example, GUI 120 may include one or more audiograms (e.g., one audiogram for each ear).

In this way, computing system 114 may determine whether a user of hearing instrument 102 perceived a sound generated by one or more audio sources 112. By determining whether the user perceived the sound, the computing system 114 may enable a hearing treatment provider to more efficiently diagnose and treat hearing impairments or disabilities. Diagnosing and treating hearing impairments or disabilities may reduce the cost of treatments and increase the quality of life of a patient.

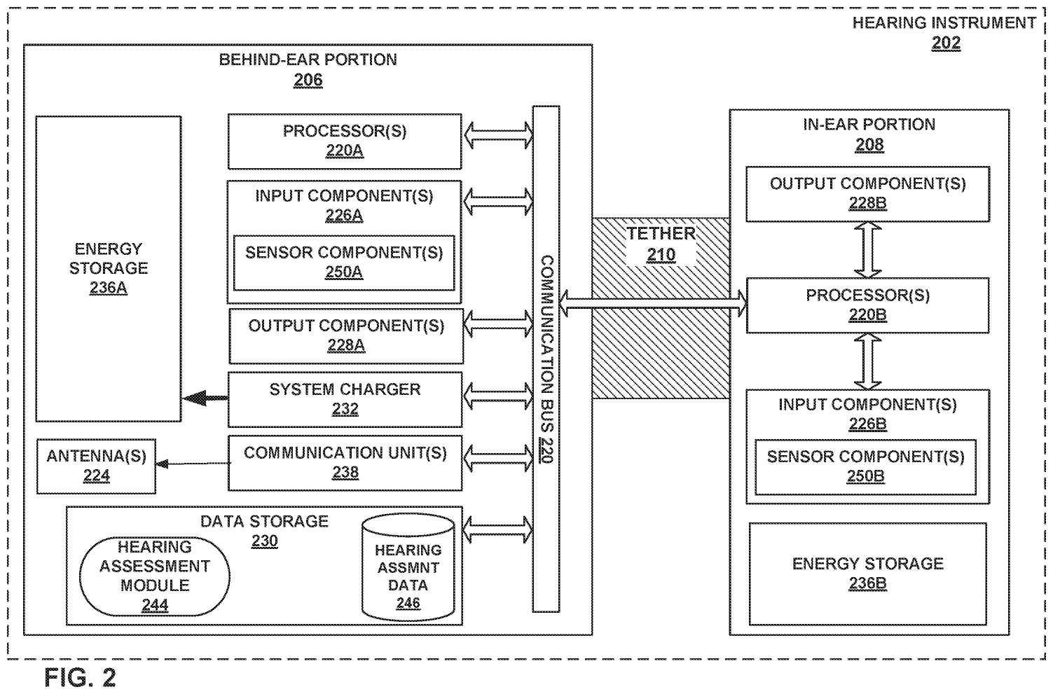

FIG. 2 is a block diagram illustrating an example of a hearing instrument 202, in accordance with one or more aspects of the present disclosure. As shown in the example of FIG. 2, hearing instrument 202 includes behind-ear portion 206 operatively coupled to in-ear portion 208 via tether 210. Hearing instrument 202, behind-ear portion 206, in-ear portion 208, and tether 210 are examples of hearing instrument 102, behind-ear portion 106, in-ear portion 108, and tether 110 of FIG. 1, respectively. It should be understood that hearing instrument 202 is only one example of a hearing instrument according to the described techniques. Hearing instrument 202 may include additional or fewer components than those shown in FIG. 2.

In some examples, behind-ear portion 206 includes one or more processors 220A, one or more antennas 224, one or more input components 226A, one or more output components 228A, data storage 230, a system charger 232, energy storage 236A, one or more communication units 238, and communication bus 240. In the example of FIG. 2, in-ear portion 208 includes one or more processors 2209, one or more input components 226B, one or more output components 228B, and energy storage 236B.

Communication bus 240 interconnects at least some of the components 220, 224, 226, 228, 230, 232, and 238 for inter-component communications. That is, each of components 220, 224, 226, 228, 230, 232, and 238 may be configured to communicate and exchange data via a connection to communication bus 240. In some examples, communication bus 240 is a wired or wireless bus. Communication bus may include a system bus, a network connection, an inter-process communication data structure, or any other method for communicating data.

Input components 226A-226B (collectively, input components 226) are configured to receive various types of input, including tactile input, audible input, image or video input, sensory input, and other forms of input. Non-limiting examples of input components 226 include a presence-sensitive input device or touch screen, a button, a switch, a key, a microphone, a camera, or any other type of device for detecting input from a human or machine. Other non-limiting examples of input components 226 include one or more sensor components 250A-250B (collectively, sensor components 250). In some examples, sensor components 250 include one or more motion sensing devices (e.g., motion sensing devices 116 of FIG. 1, such as an accelerometer, a gyroscope, a magnetometer, an inertial measurement unit (IMU), among others) configured to generate motion data indicative of motion of hearing instrument 202. The motion data may include processed and/or unprocessed data representing the motion. Some additional examples of sensor components 250 include a proximity sensor, a global positioning system (GPS) receiver or other type of location sensor, a temperature sensor, a barometer, an ambient light sensor, a hydrometer sensor, a heart rate sensor, a magnetometer, a glucose sensor, an olfactory sensor, a compass, an antennae for wireless communication and location sensing, a step counter, to name a few other non-limiting examples.

Output components 228A-228B (collectively, output components 228) are configured to generate various types of output, including tactile output, audible output, visual output (e.g., graphical or video), and other forms of output. Non-limiting examples of output components 228 include a sound card, a video card, a speaker, a display, a projector, a vibration device, a light, a light emitting diode (LED), or any other type of device for generating output to a human or machine.

One or more communication units 238 enable hearing instrument 202 to communicate with external devices (e.g., computing system 114) via one or more wired and/or wireless connections to a network (e.g., network 118 of FIG. 1). Communication units 238 may transmit and receive signals that are transmitted across network 118 and convert the network signals into computer-readable data used by one or more of components 220, 224, 226, 228, 230, 232, and 238. One or more antennas 224 are coupled to communication units 238 and are configured to generate and receive the signals that are broadcast through the air (e.g., via network 118).

Examples of communication units 238 include various types of receivers, transmitters, transceivers, BLUETOOTH® radios, short wave radios, cellular data radios, wireless network radios, universal serial bus (USB) controllers, proprietary bus controllers, network interface cards, optical transceivers, radio frequency transceivers, or any other type of device that can send and/or receive information over a network. In cases where communication units 238 include a wireless transceiver, communication units 238 may be capable of operating in different radio frequency (RF) bands (e.g., to enable regulatory compliance with a geographic location at which hearing instrument 202 is being used). For example, a wireless transceiver of communication units 238 may operate in the 900 MHz or 2.4 GHz RF bands. A wireless transceiver of communication units 238 may be a near-field magnetic induction (NFMI) transceiver, and RF transceiver, an Infrared transceiver, ultra-sonic transceiver, or other type of transceiver.

In some examples, communication units 238 are configured as wireless gateways that manage information exchanged between hearing assistance device 202, computing system 114 of FIG. 1, and other hearing assistance devices. As a gateway, communication units 238 may implement one or more standards-based network communication protocols, such as Bluetooth®, Wi-Fi®, GSM, LTE, WiMAX®, 802.1X, Zigbee®, LoRa® and the like as well as non-standards-based wireless protocols (e.g., proprietary communication protocols). Communication units 238 may allow hearing instrument 202 to communicate, using a preferred communication protocol implementing intra and inter body communication (e.g., an intra or inter body network protocol), and convert the body communications to a standards-based protocol for sharing the information with other computing devices, such as computing system 114. Whether using a body network protocol, intra or inter body network protocol, body area network protocol, body sensor network protocol, medical body area network protocol, or some other intra or inter body network protocol, communication units 238 enable hearing instrument 202 to communicate with other devices that are embedded inside the body, implanted in the body, surface-mounted on the body, or being carried near a person's body (e.g., while being worn, carried in or part of clothing, carried by hand, or carried in a bag or luggage). For example, hearing instrument 202 may cause behind-ear portion 106A to communicate, using an intra or inter body network protocol, with in-ear portion 108, when hearing instrument 202 is being worn on a user's ear (e.g., when behind-ear portion 106A is positioned behind the user's ear while in-ear portion 108 sits inside the user's ear.

Energy storage 236A-236B (collectively, energy storage 236) represents a battery (e.g., a well battery or other type of battery), a capacitor, or other type of electrical energy storage device that is configured to power one or more of the components of hearing instrument 202. In the example of FIG. 2, energy storage 236 is coupled to system charger 232 which is responsible for performing power management and charging of energy storage 236. System charger 232 may be a buck converter, boost converter, flyback converter, or any other type of AC/DC or DC/DC power conversion circuitry adapted to convert grid power to a form of electrical power suitable for charging energy storage 236. In some examples, system charger 232 includes a charging antenna (e.g., NFMI, RF, or other type of charging antenna) for wirelessly recharging energy storage 236. In some examples, system charger 232 includes photovoltaic cells protruding through a housing of hearing instrument 202 for recharging energy storage 236. System charger 232 may rely on a wired connection to a power source for charging energy storage 236.

One or more processors 220A-220B (collectively, processors 220) comprise circuits that execute operations that implement functionality of hearing instrument 202. One or more processors 220 may be implemented as fixed-function processing circuits, programmable processing circuits, or a combination of fixed-function and programmable processing circuits. Examples of processors 220 include digital signal processors, general purpose processors, application processors, embedded processors, graphic processing units (GPUs), digital signal processors (DSPs), application specific integrated circuits (ASICs), field programmable gate arrays (FPGAs), display controllers, auxiliary processors, sensor hubs, input controllers, output controllers, microcontrollers, and any other equivalent integrated or discrete hardware or circuitry configure to function as a processor, a processing unit, or a processing device.

Data storage device 230 represents one or more fixed and/or removable data storage units configured to store information for subsequent processing by processors 220 during operations of hearing instrument 202. In other words, data storage device 230 retains data accessed by module 244 as well as other components of hearing instrument 202 during operation. Data storage device 230 may, in some examples, include a non-transitory computer-readable storage medium that stores instructions, program information, or other data associated module 244. Processors 220 may retrieve the instructions stored by data storage device 230 and execute the instructions to perform operations described herein.

Data storage device 230 may include a combination of one or more types of volatile or non-volatile memories. In some cases, data storage device 230 includes a temporary or volatile memory (e.g., random access memories (RAM), dynamic random-access memories (DRAM), static random-access memories (SRAM), and other forms of volatile memories known in the art). In such a case, data storage device 230 is not used for long-term data storage and as such, any data stored by storage device 230 is not retained when power to data storage device 230 is lost. Data storage device 230 in some cases is configured for long-term storage of information and includes non-volatile memory space that retains information even after data storage device 230 loses power. Examples of non-volatile memories include magnetic hard discs, optical discs, flash memories, USB disks, or forms of electrically programmable memories (EPROM) or electrically erasable and programmable (EEPROM) memories.

One or more processors 220B may exchange information with behind-ear portion 206 via tether 210. One or more processors 220B may receive information from behind-ear portion 206 via tether 210 and perform an operation in response. For instance, processors 220A may send data to processors 220B that cause processors 220B to use output components 228B to generate sounds.

One or more processors 220B may transmit information to behind-ear portion 206 via tether 210 to cause behind-ear portion 206 to perform an operation in response. For example, processors 220B may receive an indication of an audio data stream being output from behind-ear portion 206 and in response, cause output components 228B to produce audible sound representative of the audio stream. As another example, sensor components 250B detect motion and send motion data indicative of the motion via tether 210 to behind-ear portion 206 for further processing, such as for detecting whether a user turned his or her head. For example, processors 220B may process at least a portion of the motion data and send a portion of the processed data to processors 220A, send at least a portion of the unprocessed motion data to processors 220A, or both. In this way, hearing instrument 202 can rely on additional processing power provided by behind-ear portion 206 to perform more sophisticated operations and provide more advanced features than other hearing instruments.

In some examples, processors 220A may receive processed and/or unprocessed motion data from sensor components 250B. Additionally, or alternatively, processors 220A may receive motion data from sensor components 250A of behind-ear portion 206. Processors 220 may process the motion data from sensor components 250A and/or 250B and may send an indication of the motion data (e.g., processed motion data and/or unprocessed motion data) to another computing device. For example, hearing instrument 202 may send an indication of the motion data via behind-ear portion 206 to another computing device (e.g., computing system 114) for further offline processing.

According to techniques of this disclosure, hearing instrument 202 may determine whether a user of hearing instrument 202 has perceived a sound. In some examples, hearing instrument 202 outputs the sound. For example, hearing instrument 202 may receive a command from a computing device (e.g., computing system 114 of FIG. 1) via antenna 224. For instance, hearing instrument 202 may receive a command to output sound in a supervised setting (e.g., a hearing assessment performed by a hearing treatment provider). In one example, the command includes a digital representation of the sound and hearing instrument 202 generates the sound in response to receiving the digital representation of the sound. In other words, hearing instrument 202 may present a sound stimulus to the user in response to receiving a command from a computing device to generate sound.

In one example, hearing instrument 202 may detect sound generated by one or more audio sources (e.g., audio sources 112 of FIG. 1) external to hearing instrument 202. In other words, hearing instrument 202 may detect the sound generated by a different audio source (e.g., one or more audio sources 112 of FIG. 1.) without receiving a command from a computing device. For example, hearing instrument 202 may detect sounds in an unsupervised setting rather than a supervised setting. In such examples, hearing instrument 202 may amplify portions of the sound to assist the user of hearing instrument 202 in hearing the sound.

Hearing assessment module 244 may store sound data associated with the sound within hearing assessment data 246 (shown in FIG. 2 as “hearing assmnt data 246”). In some examples, the sound data includes a timestamp that indicates a time associated with the sound. For example, the timestamp may indicate a time at which hearing instrument 202 received a command from a computing device (e.g., computing system 114) to generate a sound, a time at which the computing device sent the command, and/or a time at which hearing instrument 202 generated the sound. In another example, the timestamp may indicate a time at which hearing instrument 202 or computing system 114 detected a sound generated by an external audio source (e.g., audio sources 112, such as electronically-generated sound and/or naturally-occurring sound). The sound data may include data indicating one or more characteristics of the sound, such as intensity, frequency, or pressure. The sound data may include a transcript of the sound or data indicating one or more keywords included in the sound. For example, the sound may include a keyword, such as the name of the user of hearing instrument 202 or the name of another person or object familiar to the user.

In some instances, a user of hearing instrument 202 may turn his or her head in response to hearing or perceiving a sound generated by one or more of audio sources 112. For instance, sensor components 250 may include one or more motion sensing devices configured to detect motion and generate motion data indicative of the motion, The motion data may include unprocessed data and/or processed data representing the motion. Unprocessed data may include acceleration data indicating an amount of acceleration in one or more dimensions (e.g., x, y, and/or z-dimensions) over time or gyroscope data indicating a speed or rate of rotation in one or more dimensions over time. In some examples, the motion data may include processed data, such as a summary data indicative of the motion. For example, summary data may include data indicating a degree of head rotation (e.g., degree of pitch, yaw, and/or roll) of the user's head. In some instances, the motion data includes a timestamp associated with the motion, such as a timestamp indicating a time at which the user turned his or her head or a plurality of timestamps indicating a respective time at which respective portions of unprocessed data was received. Hearing assessment module 244 may store the motion data in hearing assessment data 246.

Heating assessment module 244 may determine whether the user perceived the sound based at least in part on the motion data and the sound data. In one example, hearing assessment module 244 determines whether the user perceived the sound based on determining whether a degree of motion of the user satisfies a motion threshold and whether an amount of time between the time associated with the sound and the time associated with the motion satisfies a time threshold.

In some examples, hearing assessment module 244 determines whether a degree of motion of the user satisfies a motion threshold. Hearing assessment module 244 may determine a degree of rotation between the initial head position and the subsequent head position based on the motion data. As one example, hearing assessment module 244 may determine the degree of rotation is approximately 45-degree (e.g., about an axis defined by the user's spine). In other words, hearing assessment module 244 may determine the user turned his or her head approximately 45-degrees. In some instances, hearing assessment module 244 compares the degree of rotation to a motion threshold to determine whether the user perceived the sound.

In some instances, hearing assessment module 244 determines the motion threshold based on hearing assessment data 246. For instance, hearing assessment data 246 may include one or more rules indicative of motion thresholds. The rules may be preprogrammed or dynamically generated (e.g., via psychometric function, machine learning). In one example, hearing assessment module 244 determines the motion threshold based on one or more characteristics of the user (e.g., age, attention span, cognition, motor function, etc.), one or more characteristics of the sound (e.g., frequency, intensity, etc.), or both.

Hearing assessment module 244 may determine whether an amount of elapsed time between the time associated with the sound and the time associated with the motion satisfies a time threshold. In some instances, hearing assessment module 244 determines the time threshold based on hearing assessment data 246. For instance, hearing assessment data 246 may include one or more rules indicative of time thresholds. The rules may be preprogrammed or dynamically generated (e.g., via psychometric function, machine learning). In one example, hearing assessment module 244 determines the time threshold based on one or more characteristics of the user (e.g., age, attention span, cognition, motor function, etc.).

In one example, hearing instrument 202 receives a command to generate a sound from an external computing device (e.g., a computing device external to hearing instrument 202) and hearing assessment module 244 determines an elapsed time between when hearing instrument 202 generates the sound when the user turned his or her head. In one example, hearing instrument 202 detects a sound (e.g., rather than being instructed to generate a sound by a computing device external to the hearing instrument 202) and hearing assessment module 244 determines the elapsed time between when hearing instrument 202 detected the sound and when the user turned his or her head.

Hearing assessment module 244 may selectively determine the elapsed time between a sound and the user's head motion. In some scenarios, hearing assessment module 244 determines the elapsed time in response to determining one or more characteristics of the sound correspond to a pre-determined characteristic (e.g., frequency, intensity, keyword). For example, hearing instrument 202 may determine an intensity of the sound and may determine whether the intensity satisfies a threshold intensity. For example, a user may be more likely to turn his or her head when the sound is relatively loud. In such examples, hearing assessment module 244 may determine whether the elapsed time satisfies a time threshold in response to determining the intensity of the sound satisfies the threshold intensity.

In another scenario, hearing assessment module 244 determines a change in the intensity of the sound and compares to a threshold change in intensity. For instance, a user may be more likely to turn his or her head when the sound is at least a threshold amount louder than the current sound. In such scenarios, hearing assessment module 244 may determine whether elapsed time satisfies the time threshold in response to determining the change in intensity of the sound satisfies a threshold change in intensity.

As vet another example, example, the pre-determined characteristic includes a particular keyword. Hearing assessment module 244 may determine whether the sound includes the keyword. For instance, a user of hearing instrument 202 may be more likely to turn his or her head when the sound includes a keyword, such as his or her name or the name of a particular object (e.g., “ball”, “dog”, “mom”, “dad”, etc.). Hearing assessment module 244 may determine whether the elapsed time satisfies the time threshold in response to determining the sound includes the particular keyword.

Hearing assessment module 244 may determine that the user did not perceive the sound in response to determining that the degree of rotation does not satisfy (e.g., is less than) a motion threshold. For instance, if the user does not turn his or her head at least a threshold amount, this may indicate the sound was not the reason that the user moved his or her head. Similarly, hearing assessment module 244 may determine that the user did not perceive the sound in response to determining that the amount of elapsed time satisfies (e.g., is greater than or equal to) a time threshold. For instance, if the user does not turn his or her head within a threshold amount of time from when the sound occurred, this may indicate the sound was not the reason that the user moved his or her head.

Hearing assessment module 244 may determine that the user perceived the sound in response to determining that the degree of rotation satisfies (e.g., is greater than) a motion threshold and that the amount of elapsed time does not satisfy (e.g., is less than) the time threshold. In other words, if the user turns his or her head at least a threshold amount within the time threshold of the sound occurring, hearing assessment module 244 may determine the user perceived the sound.

Additionally, or alternatively, hearing assessment module 244 may determine whether the user perceived the sound based on a direction in which the user turned his or her head. Hearing assessment module 244 may determine the motion direction based on the motion data. For example, hearing assessment module 244 may determine whether the user turned his or her head left or right. In some examples, hearing assessment module 244 determines whether the user perceived the sound based on whether the user turned his or her head in the direction of the audio source 112 that generated the sound.

Hearing assessment module 244 may determine a direction of the source of the sound relative to the user. In one example, hearing instrument 202 may be associated with a particular ear of the user (e.g., either the left ear or the right ear) and may receive a command to output the sound, such that hearing assessment module 244 may determine the direction of the audio based on the ear associated with hearing instrument 202. For instance, hearing instrument 202 may determine that hearing instrument 202 is associated with (e.g., worn on or in) the user's left ear and may output the sound, such that hearing assessment module 244 may determine the direction of the source of the sound is to the left of the user.

In some examples, hearing assessment module 244 determines a direction of the source (e.g., one or more audio sources 112 of FIG. 1) of the sound relative to the user based on data received from another hearing instrument. For example, hearing instrument 202 may be associated with one ear of the user (e.g., the user's left ear) and another hearing instrument may be associated with the other ear of the user (e.g., the user's right ear). Hearing assessment module 244 may receive sound data from another hearing instrument 202 and may determine the direction of the source of the sound based on the sound data from both hearing instruments (e.g., hearing instrument 202 associated with the user's left ear and the other hearing instrument associated with the user's right ear). In one example, hearing assessment module 244 may determine the direction of the source of the sound based on one or more characteristics of the sound (e.g., intensity level at each ear and/or time at which the sound was detected). For example, hearing assessment module 244 may determine the direction of the source of the sound corresponds to the direction of hearing instrument 202 (e.g., the sound came from the left of the user) response to determining the sound detected by hearing instrument 202 was louder than sound detected by the other hearing instrument.

Additionally, or alternatively, hearing assessment module 344 may determine the direction of the source of the sound based on a time at which hearing instruments 202 detect the sound. For example, hearing assessment module 344 may determine a time at which the sound was detected by hearing instrument 202. Hearing assessment module 344 may determine a time at which the sound was detected by another hearing instrument based on sound data received from the other hearing instrument. In some instances, hearing assessment module 344 determines the direction of the source corresponds to the side of the user's head that is associated with hearing instrument 202 in response to determining that hearing instrument 202 detected the sound prior to another hearing instrument associated with the other side of the user's head. In other words, hearing assessment module 344 may determine that the source of the sound is located to the right of the user in response to determining that the hearing instrument 202 associated with the right side of the user's head detected the sound before the hearing instrument associated with the left side of the user's head.

Responsive to determining the direction of source of the sound relative to the user, hearing assessment module 244 may determine the user perceived the sound in response to determining the user moved his or her head in the direction of source of the sound (e.g., in the direction of one or more audio sources 112). Hearing assessment module 244 may determine the user did not perceive the sound in response to determining the user moved his or her head in a direction different than the direction of the source of the sound. In other words, hearing assessment module 244 may determine whether the user perceived the sound based on whether the direction of the motion is aligned with the direction of audio source 112. In one example, hearing assessment module 244 determines the user perceived the sound in response to determining the direction of motion is aligned with the direction of audio source 112. In another example, hearing assessment module 244 determines the user did not perceive the sound in response to determining the direction of the motion is not aligned with the direction of the sound.

Hearing assessment module 244 may store analysis data indicating whether the user perceived the sound in hearing assessment data 246. In some examples, the analysis data includes a summary of characteristics of sounds perceived by the user and/or sound sounds not perceived by the user. For example, the analysis data may indicate which frequencies of sound were or were not detected, which intensity levels of sound were or were not detected, the locations of the sounds that were or were not detected, or a combination thereof.

Responsive to determining whether the user perceived the sound, hearing assessment module 244 may output all or a portion of the analysis data indicating whether the user perceived the sound. In one example, hearing assessment module 244 outputs analysis data to another computing device (e.g., computing system 114 of FIG. 1) via communication units 238 and antenna 224. Additionally, or alternatively, hearing assessment data may output all or portions of the sound data and/or the motion data to computing system 114.

In this way, hearing assessment module 244 of hearing instrument 202 may determine whether a user of hearing instrument 202 perceived a sound. Utilizing hearing instrument 202 to determine whether a user perceived the sound may reduce data transferred to another computing device, such as computing system 114 of FIG. 1, which may reduce battery power consumed by hearing instrument 202. Hearing assessment module 244 may determine whether the user perceived sounds without receiving a command to generate the sounds from another computing device, which may enable hearing assessment module 244 to assess the hearing of a user of hearing instrument 202 in an unsupervised setting rather than a supervised, clinical setting. Assessing hearing of the user in an unsupervised setting may enable hearing assessment module 244 to more accurately determine the characteristics of sounds that can be perceived by the user in everyday environment rather than a test environment.

While hearing assessment module 244 is described as determining whether the user perceived the sound, in some examples, part or all of the functionality of hearing assessment module 244 may be performed by another computing device (e.g., computing system 114 of FIG. 1). For example, hearing assessment module 244 may output all or a portion of the sound data and/or the motion data to computing system 114 such that computing system 114 may determine whether the user perceived the sound or assist hearing assessment module 244 in determining whether the user perceived the sound.

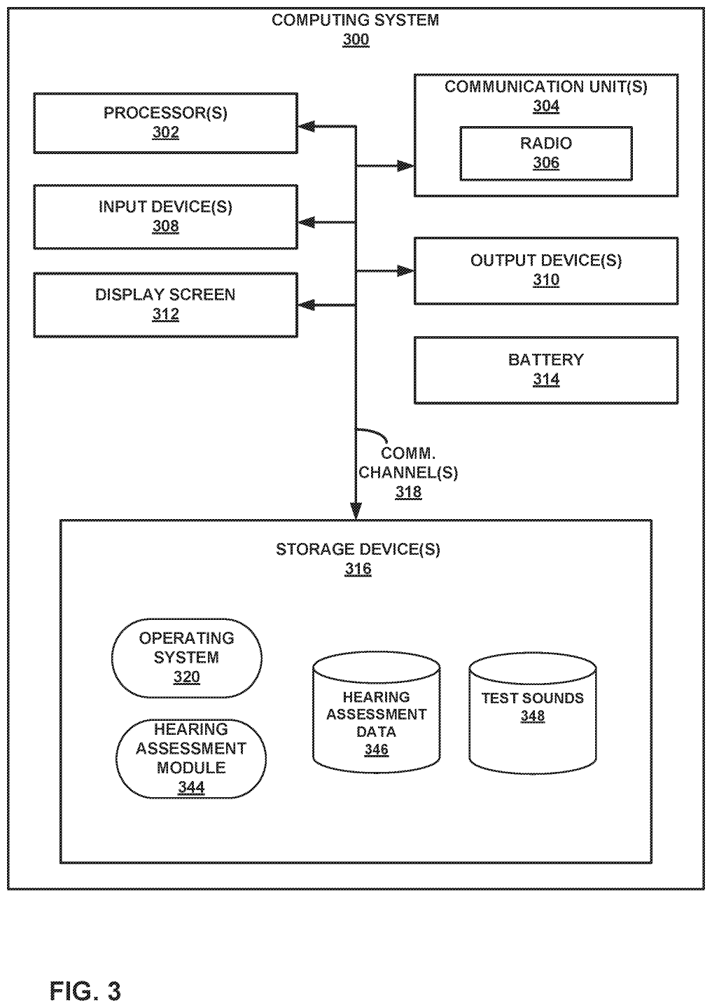

FIG. 3 is a block diagram illustrating example components of computing system 300, in accordance with one or more aspects of this disclosure. FIG. 3 illustrates only one particular example of computing system 300, and many other example configurations of computing system 300 exist. Computing system 300 may be a. computing system in computing system 114 (FIG. 1). For instance, computing system 300 may be mobile computing device, a laptop or desktop computing device, a distributed computing system, or any other type of computing system.

As shown in the example of FIG. 3, computing system 300 includes one or more processors 302, one or more communication units 304, one or more input devices 308, one or more output devices 310, a display screen 312, a battery 314, one or more storage devices 316, and one or more communication channels 318. Computing system 300 may include many other components. For example, computing system 300 may include physical buttons, microphones, speakers, communication ports, and so on. Communication channel(s) 318 may interconnect each of components 302, 304, 308, 310, 312, and 316 for inter-component communications (physically, communicatively, and/or operatively). In some examples, communication channel(s) 318 may include a system bus, a network connection, an inter-process communication data structure, or any other method for communicating data. Battery 314 may provide electrical energy to one or more of components 302, 304. 308, 310, 312 and 316.

Storage device(s) 316 may store information required for use during operation of computing system 300. In some examples, storage device(s) 316 have the primary purpose of being a short term and not a long-term computer-readable storage medium. Storage device(s) 316 may be volatile memory and may therefore not retain stored contents if powered off. Storage device(s) 316 may further be configured for long-term storage of information as non-volatile memory space and retain information after power on/off cycles. In some examples, processor(s) 302 on computing system 300 read and may execute instructions stored by storage device(s) 316.

Computing system 300 may include one or more input device(s) 308 that computing system 300 uses to receive user input. Examples of user input include tactile, audio, and video user input. Input device(s) 308 may include presence-sensitive screens, touch-sensitive screens, mice, keyboards, voice responsive systems, microphones or other types of devices for detecting input from a human or machine.

Communication unit(s) 304 may enable computing system 300 to send data to and receive data from one or more other computing devices (e.g., via a communications network, such as a local area network or the Internet). In some examples, communication unit(s) 304 may include wireless transmitters and receivers that enable computing system 300 to communicate wirelessly with the other computing devices. For instance, in the example of FIG. 3, communication unit(s) 304 include a radio 306 that enables computing system 300 to communicate wirelessly with other computing devices, such as hearing instrument 102, 202 of FIGS. 1, 2, respectively. Examples of communication unit(s) 304 may include network interface cards, Ethernet cards, optical transceivers, radio frequency transceivers, or other types of devices that are able to send and receive information. Other examples of such communication units may include Bluetooth, 3G, and WIFI radios, Universal Serial Bus (USB) interfaces, etc. Computing system 300 may use communication unit(s) 304 to communicate with one or more hearing instruments 102, 202. Additionally, computing system 300 may use communication unit(s) 304 to communicate with one or more other remote devices (e.g., audio sources 112 of FIG. 1).

Output device(s) 310 may generate output. Examples of output include tactile, audio, and video output. Output device(s) 310 may include presence-sensitive screens, sound cards, video graphics adapter cards, speakers, liquid crystal displays (LCD), or other types of devices for generating output.

Processor(s) 302 may read instructions from storage device(s) 316 and may execute instructions stored by storage device(s) 316. Execution of the instructions by processor(s) 302 may configure or cause computing system 300 to provide at least some of the functionality ascribed in this disclosure to computing system 300. As shown in the example of FIG. 3, storage device(s) 316 include computer-readable instructions associated with operating system 320 and hearing assessment module 344. Additionally, in the example of FIG. 3, storage device(s) 316 may store hearing assessment data 346.

Execution of instructions associated with operating system 320 may cause computing system 300 to perform various functions to manage hardware resources of computing system 300 and to provide various common services for other computer programs.

Execution of instructions associated with hearing assessment module 344 may cause computing system 300 to perform one or more of various functions described in this disclosure with respect to computing system 114 of FIG. 1 and/or hearing instruments 102, 202 of FIGS. 1, 2, respectively. For example, execution of instructions associated with hearing assessment module 344 may cause computing system 300 to configure radio 306 to wirelessly send data to other computing devices (e.g., hearing instruments 102, 202, or audio sources 112) and receive data from the other computing devices. Additionally, execution of instructions of hearing assessment module 344 may cause computing system 300 to determine whether a user of a hearing instrument 102, 202 perceived a sound.

A user of computing system 300 may initiate a hearing assessment test session to determine whether a user of a hearing instrument 102, 202 perceives a sound. For example, computing system 300 may execute hearing assessment module 344 in response to receiving a user input from a hearing treatment provider to begin the hearing assessment. As another example, computing system 300 may execute hearing assessment module 344 in response to receiving a user input from a user of hearing instrument 102, 202 (e.g., a patient).

Hearing assessment module 344 may output a command to one or more one or more electronic devices that include a speaker (e.g., audio sources 112 of FIG. 1 and/or hearing instruments 102, 202) to cause the speaker to generate sound. In some instances, hearing assessment module 344 may output a plurality of commands, for instance, to different audio sources 112 and/or hearing instruments 102, 202. For instance, hearing assessment module 344 may output a first command to a hearing instrument 102, 202 associated with one ear, a second command to a hearing instrument associated with the user's other ear, and/or a third command to a plurality of hearing instruments associated with both ears.

In some examples, hearing assessment module 344 outputs a command to generate sound, the command including a digital representation of the sound. For instance, test sounds 348 may include digital representations of sound and the command may include one or more of the digital representations of sound stored in test sounds 348. In other examples, hearing assessment 344 may stream the digital representation of the sound from another computing device or cause an audio source 112 or hearing instrument 102, 202 to retrieve the digital representation of the sound from another source (e.g., an interact sound provider, such as an interact music provider). In some instances, hearing assessment module 344 may control the characteristics of the sound, such as the frequency, bandwidth, modulation, phase, and/or level of the sound.

Hearing assessment module 344 may output a command to generate sounds from virtual locations around the user's head. For example, hearing assessment module 344 may estimate a virtual location in space around the user at which to present the sound utilizing a Head-Related Transfer Function (HRTF). In one example, hearing assessment module 344 estimates the virtual location based at least in part on the head size of the listener. In another example, hearing assessment module 344 may include an individualized HRTF associated with the user (e.g., the patient).

According to one example, the command to generate sound may include a command to generate sounds from “static” virtual locations. As used throughout this disclosure, a static virtual location means that the apparent location of the sound in space does not change when the user turns his or her head. For instance, if sounds are presented to the left of the user, and the user turns his or her head to the right, sounds will now be perceived to be from behind the listener. As another example, the command to generate sound may include a command to generate sound from “dynamic” or “relative” virtual locations. As used throughout this disclosure, a dynamic or relative virtual location means the location of the sound follows the user's head. For instance, if sounds are presented to the left of the user and the user turns his or her head to the right, the sounds will still be perceived to be from the left of the listener.

In one scenario, hearing assessment module 344 may determine whether to utilize a static or dynamic virtual location based on characteristics of the user, such as age, attention span, cognition or motor function. For example, an infant or other individual may have limited head control and may be unable to center his or her head. In such examples, hearing assessment module 344 may determine to output a command to generate sound from dynamic virtual locations.

Hearing assessment module 344 may determine one or more characteristics of the sound generated by hearing instrument 102, 202 or audio sources 112. Examples of the characteristics of the sound include the sound frequency, intensity level, location (or apparent or virtual location) of the source of the sound, amount of time between sounds, among others. In one example, hearing assessment module 344 determines the characteristics of the sound based on whether the user perceived a previous sound.

For example, hearing assessment module 344 may output a command to alter the intensity level (e.g., decibel level) of the sound based on whether the user perceived a previous sound. As one example, hearing assessment module 344 may utilize an adaptive method to control the intensity level of the sound. For instance, hearing assessment module 344 may cause hearing instrument 102, 202, or audio sources 112 to increase the volume in response to determining the user did not perceive a previous sound or lower the volume in response to determining the user did perceive a previous sound. In one scenario, the command to generate sound includes a command to increase the intensity level by a first amount (e.g., 10 dB) if the user did not perceive the previous sound and decrease the intensity level by another (e.g., different) amount (e.g., 5 dB) in response to determining the user did perceive the previous sound.

In another example, hearing assessment module 344 may determine the time between when sounds are generated. In some examples, hearing assessment module 344 determines the time between sounds based on a probability the user perceived a. previous sound. For example, hearing assessment module 344 may determine the probability the user perceived the previous sound based at least in part on a degree of rotation of the user's head (e.g., assigning a higher probability as the degree of rotation associated with the previous sound increases). As another example, hearing assessment module 344 may determine the probability the user perceived the previous sound based at least in part on the amount of time between an amount of elapsed time between the time associated with the sound and the time associated with the motion (e.g., assigning a lower probability as the elapsed time associated with the previous sound increases).

In one example, hearing assessment module 344 may determine to output a subsequent sound relatively quickly after determining the probability the user perceived a previous sound was relatively high (e.g., 80%). As another example, hearing assessment module 344 may determine to output the subsequent sound after a relatively long amount of time in response to determining the probability the user perceived the previous sound was relatively low (e.g., 25%), which may provide the user with more time to move his or her head. In some scenarios, hearing assessment module 344 determines the time between sounds is a pre-defined amount of time or a random amount of time.

Hearing assessment module 344 may determine whether a user perceived a. sound based at least in part on data from a hearing instrument 102, 202. In some examples, hearing assessment module 344 may request analysis data, sound data, and/or motion data) from hearing instrument 102, 202 for determining whether the user perceived a sound. Hearing assessment module 344 may request the data periodically (e.g., every 30 minutes) or in response to receiving an indication of user input requesting the data. In some examples, hearing instrument 102, 202 pushes the analysis, motion, and/or sound data to computing system 300. For example, hearing instrument 102 may push the data to computing device 300 in response to detecting sound, in response to determining the user did not perceive the sound, or in response to determining the user did perceive the sound, as some examples. In some examples, exchanging data between hearing instrument 102, 202 and computing system 300 when computing system 300 receives an indication of user input requesting the hearing assessment data, or upon determining the user did or did not perceive a particular sound, may reduce demands on a battery of hearing instrument 102, 202 relative to computing system 300 requesting the data from hearing instrument 102, 202 on a periodic basis.

In some examples, hearing assessment module 344 receives motion data from hearing instrument 102, 202. As another example, hearing assessment module 344 may receive sound data from hearing instrument 102, 202. For instance, a hearing instrument 102, 202 may detect sounds in the environment that are not caused by an electronic device (e.g., sounds that are not generated in response to a command from computing device 300) and may output sound data associated with the sounds to computing device 300. Hearing assessment module 344 may store the motion data and/or sound data in hearing assessment data 346. Hearing assessment module 344 may determine whether the user perceived the sound in a manner similar to the techniques for hearing instruments 102, 202, or computing system 114 described above. In some examples, hearing assessment module 344 may store analysis data indicative of whether the user perceived the sound within hearing assessment data 346. For instance, the analysis data may indicate which frequencies of sound were or were not detected, which decibel levels of sound were or were not detected, the locations of the sounds that were or were not detected, or a combination thereof. In this way, hearing assessment module 344 may determine whether the user perceived the sound whether the sound was generated in response to a command from computing device 300 or was a naturally occurring sound. For instance, hearing assessment module 344 may perform a hearing assessment in a supervised setting and/or an unsupervised setting.

Responsive to determining whether the user perceived the sound, hearing assessment module 344 may output data indicating whether the user perceived the sound. In one example, hearing assessment module 344 outputs analysis data to another computing device (e.g., a computing device associated with a hearing treatment provider). Additionally, or alternatively, hearing assessment data may output all or portions of the sound data and/or the motion data. In some instances, hearing assessment module 344 outputs a GUI that includes all or a portion of the analysis data. For instance, the GUI may indicate which frequencies of sound were or were not detected, which decibel levels of sound were or were not detected, the locations of the sounds that were or were not detected, or a combination thereof. In some examples, the GUI includes one or more audiograms (e.g., one audiogram for each ear).

Hearing assessment module 344 may output data indicative of a reward for the user in response to determining the user perceived the sound. In one example, the data indicative of the reward include data associated with an audible or visual reward. For example, hearing assessment module 344 may output a command to a display device to display an animation (e.g., congratulating or applauding a child for moving his or her head) and/or a command to hearing instrument 102, 202 to generate a sound (e.g., a sound that includes praise words for the child). In this way, hearing assessment module 344 may help teach the user to turn his or her head when he or she hears a sound, which may improve the ability to detect user's head motion and thus determine whether the user moved his or her head in response to perceiving the sound.

In some scenarios, hearing assessment module 344 may output data to a remote computing device, such as a computing device associated with a hearing treatment provider. For example, computing device 300 may include a camera that generates image data (e.g., pictures and/or video) of the user and transmits the image data to the hearing treatment provider. In this way, computing device 300 may enable a telehealth hearing assessment with a hearing treatment provider and enable to hearing treatment provider to more efficiently diagnose and treat hearing impairments or disabilities.

Utilizing computing system 300 to determine whether a user perceived a sound may reduce the computations performed by hearing instrument 102, 202. Reducing the computations performed by hearing instrument 102, 202 may increase the battery life of hearing instrument 102, 202 or enable hearing instrument 102, 202 to utilize a smaller battery. Utilizing a smaller battery may increase space for additional components within hearing instrument 102, 202 or reduce the size of hearing instrument 102, 202.

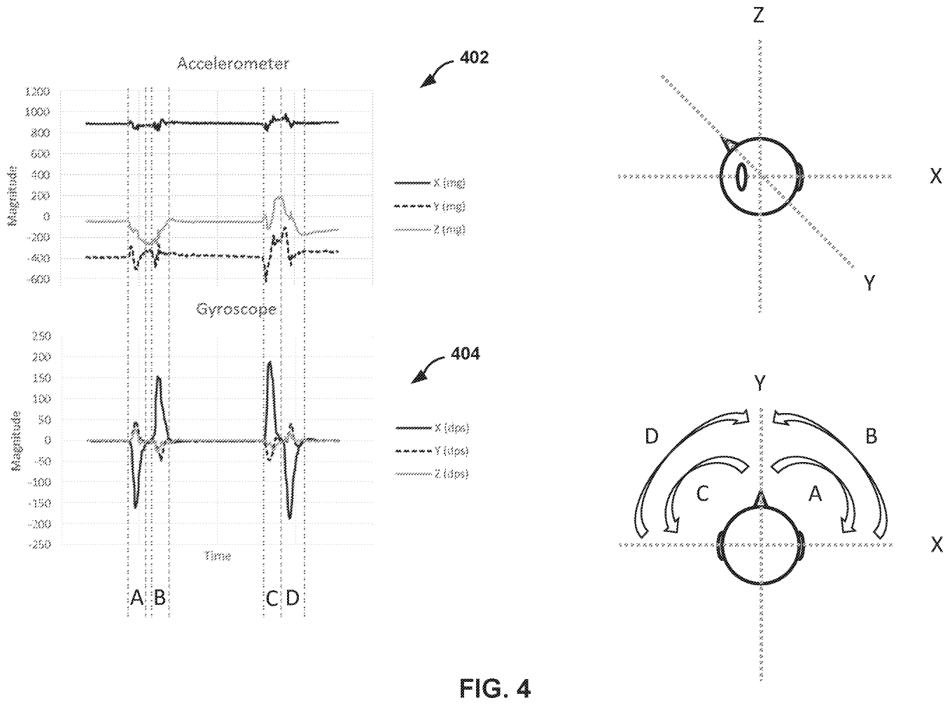

FIG. 4 illustrates graphs of example motion data, in accordance with one or more aspects of the present disclosure. The motion data is associated with four distinct head turns. For example, head turn A represents a turn from approximately 0-degrees (e.g., straight forward) to approximately 90-degrees (e.g., turning the head to the right). Head turn B represents a turn from approximately 90-degrees to approximately 0-degrees. Head turn C represents a turn from approximately 0-degrees to approximately negative (−) 90-degrees (e.g., turn the head to the left). Head turn D represents a turn from approximately negative 90-degrees to approximately 0-degrees.

Graph 402 illustrates an example of motion data generated by an accelerometer. As illustrated in graph 402, during head turns A-D, the accelerometer detected relatively little motion in the x-direction. However, as also illustrated in graph 402, the accelerometer detected relatively larger amounts or degrees of motion in the y-direction and the z-direction as compared to the motion in the x-direction.

Graph 404 illustrates an example of motion data generated by a gyroscope. As illustrated in graph 404, the gyroscope detected relatively large amounts of motion in the x-direction during head turns A-D. As further illustrated by graph 404, the gyroscope detected relatively small amounts of motion in the y-direction and z-direction relative to the amount of motion in the x-direction.

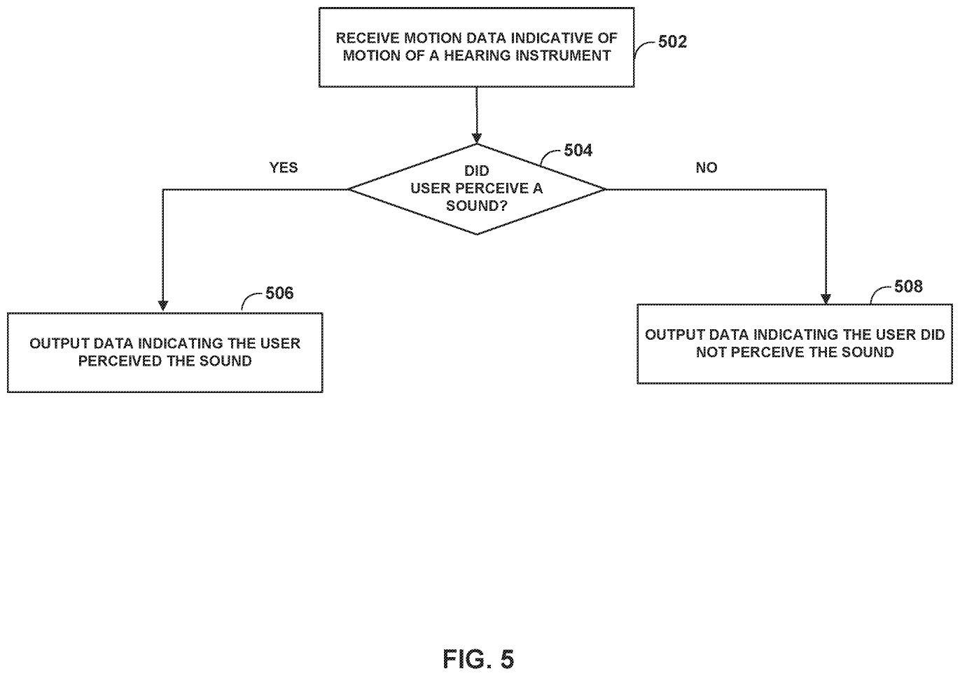

FIG. 5 is a flowchart illustrating an example operation of computing system 114, in accordance with one or more aspects of this disclosure. The flowcharts of this disclosure are provided as examples. In other examples, operations shown in the flowcharts may include more, fewer, or different actions, or actions may be performed in different orders or in parallel.

In the example of FIG. 5, computing system 114 receives motion data indicative of motion of a hearing instrument 102 (502). The motion data may include processed motion data and/or unprocessed motion data.

Computing system 114 determines whether a user of hearing instrument 102 perceived a sound (504). In one example, computing system 114 outputs a command to hearing instrument 102 or audio sources 112 to generate the sound. In another example, the sound is a sound occurring in the environment rather than a sound caused by an electronic device receiving a command from computing system 114. In some scenarios, computing system 114 determines whether the user perceived the sound based on the motion data. For example, computing system 114 may determine a degree of motion of the user's head based on the motion data. Computing system 114 may determine that the user perceived the sound in response to determining the degree of motion satisfies a motion threshold. In one instance, computing system 114 determines that the user did not perceive the sound in response to determining that the degree of motion does not satisfy the motion threshold.

In another scenario, computing system 114 determines whether the user perceived the sound based on the motion data and sound data associated with the sound. The motion data may indicate a time associated with the motion, such as a timestamp indicating a time at which the user turned his or her head or a plurality of timestamps indicating a respective time at which various portions of unprocessed data was received. The sound data may include a timestamp that indicates a time associated with the sound. The time associated with the sound may include a time at which computing system 114 output a command to generate the sound, a time at which the sound was generated, or a time at which the sound was detected by hearing instrument 102. In some instances, computing system 114 determines an amount of elapsed time between the time associated with the sound and the time associated with the motion. Computing system 114 may determine that the user perceived the sound in response to determining that the degree of motion satisfies (e.g., is greater than or equal to) the motion threshold and that the elapsed time does not satisfy (e.g., is less than) a time threshold. In one example, computing system 114 determines that the user did not perceive the sound in response to determining that the degree of motion does not satisfy the motion threshold and/or that the elapsed time satisfies a time threshold.

Computing system 114 may output data indicating that the user perceived the sound (506) in response to determining that the user perceived the sound (“YES” path of 504). For example, computing system 114 may output a GUI for display by a display device that indicates an intensity level of the sound perceived by the user, a frequency of the sound perceived by the user, a location (e.g., actual location or virtual location) of the source of the sound perceived by the user, or a combination thereof.

Computing system 114 may output data indicating that the user did not perceive the sound (508) in response to determining that the user did not perceive the sound (“NO” path of 504). For example, the GUI output by computing system 114 may indicate an intensity level of the sound that is not perceived by the user, a frequency of the sound that is not perceived by the user, a location (e.g., actual location or virtual location) of the source of the sound that is not perceived by the user, or a combination thereof.

While computing system 114 is described as performing the operations to determine whether the user perceived the sound, in some examples, one or more hearing instruments 102 may perform one or more of the operations. For example, hearing instrument 102 may detect sound and determine whether the user perceived the sound based on the motion data.

The following is a non-limiting list of examples that are in accordance with one or more techniques of this disclosure.

Example 1A. A computing system comprising; a memory configured to store motion data indicative of motion of a hearing instrument; and at least one processor configured to: determine, based on the motion data, whether a user of the hearing instrument perceived a sound; and responsive to determining whether the user perceived the sound, output data indicating whether the user perceived the sound.

Example 2A. The computing system of example 1A, wherein the at least one processor is configured to determine whether the user of the hearing instrument perceived the sound by at least being configured to: determine, based on the motion data, a degree of rotation of a head of the user; determine whether the degree of rotation satisfies a motion threshold; and determine the user perceived the sound in response to determining the degree of rotation satisfies the motion threshold.

Example 3A. The computing system of example 2A, wherein the at least one processor is configured to determine the motion threshold based on one or more characteristics of the user.

Example 4A. The computing system of any one of examples 2A-3A, wherein the at least one processor is configured to determine the motion threshold based on one or more characteristics of the sound.

Example 5A. The computing system of any one of examples 1A-4A, wherein the at least one processor is further configured to: receive sound data indicating a time at which the sound was detected by the hearing instrument, wherein execution of the instructions causes the at least one processor to determine whether the user perceived the sound further based on the time at which the sound was detected by the hearing instrument.

Example 6A. The computing system of example 5A, wherein the at least one processor is configured to determine whether the user perceived the sound by at least being configured to: determine, based on the motion data, a time at which the user turned a head of the user; determine an amount of elapsed time between the time at which the user turned the head of the user and the time at which the sound was detected, and determine the user perceived the sound in response to determining the amount of elapsed time does not satisfy a time threshold.

Example 7A. The computing system of example 6A, wherein the at least one processor is configured to determine the time threshold based on one or more characteristics of the user.

Example 8A. The computing system of any one of examples 1A-7A, wherein the at least one processor is configured to determine whether the user of the hearing instrument perceived the sound based at least in part on a direction the user turned a head of the user.

Example 9A. The computing system of example 8A, wherein the at least one processor is further configured to: determine, based on one or more characteristics of the sound, a direction of an audio source that generated the sound; and determine that the user perceived the sound in response to determining that the direction the user turned the head is aligned with the direction of the audio source.

Example 10A. The computing system of example 9A, wherein the hearing instrument is a first hearing instrument, and wherein the at least one processor is configured to determine a direction of the audio source was received by at least being configured to: receive first sound data from the first hearing instrument; receive second sound data from a second hearing instrument; determine the direction of the audio source based on the first sound data and the second sound data.

Example 11A. The computing system of any one of examples 1A-10A, wherein the computing system comprises the hearing instrument, wherein the hearing instrument includes the memory and the at least one processor.

Example 12A. The computing system of any one of examples 1A-10A, further comprising a computing device physically distinct from the hearing instrument, the computing device comprising the memory and the at least one processor.

Example 1B. A method comprising: receiving, by at least one processor, motion data indicative of motion of a hearing instrument; determining, by the at least one processor, based on the motion data, whether a user of the hearing instrument perceived a sound; and responsive to determining whether the user perceived the sound, outputting, by the one or more processors, data indicating whether the user perceived the sound.

Example 2B. The method of example 1B, wherein determining whether the user of the hearing instrument perceived the sound comprises: determining, by the at least one processor, based on the motion data, a degree of rotation of a head of the user; determining, by the at least one processor, whether the degree of rotation satisfies a motion threshold; and determining, by the at least one processor, that the user perceived the sound in response to determining the degree of rotation satisfies the motion threshold.

Example 3B. The method of example 2B, wherein determining the motion threshold is based on one or more characteristics of the user or one or more characteristics of the sound.

Example 4B. The method of any one of examples 1B-3B, further comprising: receiving, by the at least one processor, sound data indicating a time at which the sound was detected by the hearing instrument, wherein determining whether the user perceived the sound is further based on the time at which the sound was detected by the hearing instrument.

Example 5B. The method of example 4B, wherein determining whether the user perceived the sound comprises: determining, by the at least one processor, based on the motion data, a time at which the user turned a head of the user; determining, by the at least one processor, an amount of elapsed time between the time at which the user turned the head of the user and the time at which the sound was detected; and determining, by the at least one processor, that the user perceived the sound in response to determining the amount of elapsed time does not satisfy a time threshold.

Example 6B. The method of any one of examples 1B-5B, wherein determining whether the user of the hearing instrument perceived the sound is based at least in part on a direction the user turned a head of the user.

Example 7B. The method of example 6B, further comprising: determining, by the at least one processor, based on one or more characteristics of the sound, a direction of an audio source that generated the sound; and determining, by the at least one processor, that the user perceived the sound in response to determining that the direction the user turned the head is aligned with the direction of the audio source.

Example 1C. A computer-readable storage medium comprising instructions that, when executed by at least one processor of a computing device, cause the at least one processor to: receive motion data indicative of motion of a hearing instrument; determine, based on the motion data, whether a user of the hearing instrument perceived a sound; and responsive to determining whether the user perceived the sound, output data indicating whether the user perceived the sound.

Example 1D. A system comprising means for performing the method of any of examples 1B-7B.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection may be considered a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transitory, tangible storage media. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. Processing circuits may be coupled to other components in various ways. For example, a processing circuit may be coupled to other components via an internal device interconnect, a wired or wireless network connection, or another communication medium.

Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

1. A computing system comprising:
a memory configured to store motion data indicative of motion of a hearing instrument; and
at least one processor configured to:
determine, based on the motion data, whether a user of the hearing instrument perceived a sound, wherein the at least one processor is configured to determine whether the user of the hearing instrument perceived the sound by at least being configured to:
determine, based on the motion data, a degree of rotation of a head of the user;
determine a motion threshold based on at least one of age of the user, attention span of the user, cognition of the user, or motor function of the user;
determine whether the degree of rotation satisfies the motion threshold; and
determine the user perceived the sound in response to determining the degree of rotation satisfies the motion threshold; and
responsive to determining whether the user perceived the sound, output data indicating whether the user perceived the sound.

2-3. (canceled)

4. The computing system of claim 1, wherein the at least one processor is configured to determine the motion threshold based on one or more characteristics of the sound.

5. The computing system of claim 1, wherein the at least one processor is further configured to:
receive sound data indicating a time at which the sound was detected by the hearing instrument,
wherein execution of the instructions causes the at least one processor to determine whether the user perceived the sound further based on the time at which the sound was detected by the hearing instrument.

6. The computing system of claim 5, wherein the at least one processor is configured to determine whether the user perceived the sound by at least being configured to:
determine, based on the motion data, a time at which the user turned a head of the user;
determine an amount of elapsed time between the time at which the user turned the head of the user and the time at which the sound was detected, and
determine the user perceived the sound in response to determining the amount of elapsed time does not satisfy a time threshold.

7. The computing system of claim 6, wherein the at least one processor is configured to determine the time threshold based on one or more characteristics of the user.

8. The computing system of claim 1, wherein the at least one processor is configured to determine whether the user of the hearing instrument perceived the sound based at least in part on a direction the user turned a head of the user.

9. The computing system of claim 8, wherein the at least one processor is further configured to:
determine, based on one or more characteristics of the sound, a direction of an audio source that generated the sound; and
determine that the user perceived the sound in response to determining that the direction the user turned the head is aligned with the direction of the audio source.

10. The computing system of claim 9, wherein the hearing instrument is a first hearing instrument, and wherein the at least one processor is configured to determine a direction of the audio source was received by at least being configured to:
receive first sound data from the first hearing instrument;
receive second sound data from a second hearing instrument; and
determine the direction of the audio source based on the first sound data and the second sound data.

11. The computing system of claim 1, wherein the computing system comprises the hearing instrument, wherein the hearing instrument includes the memory and the at least one processor.

12. The computing system of claim 1, further comprising a computing device physically distinct from the hearing instrument, the computing device comprising the memory and the at least one processor.

13. A method comprising:
receiving, by at least one processor, motion data indicative of motion of a hearing instrument;
determining, by the at least one processor, based on the motion data, whether a user of the hearing instrument perceived a sound, wherein determining whether the user of the hearing instrument perceived the sound comprises:
determining, based on the motion data, a degree of rotation of a head of the user;
determining a motion threshold based on at least one of age of the user, attention span of the user, cognition of the user, or motor function of the user;
determining whether the degree of rotation satisfies the motion threshold; and
determining the user perceived the sound in response to determining the degree of rotation satisfies the motion threshold; and
responsive to determining whether the user perceived the sound, outputting, by the one or more processors, data indicating whether the user perceived the sound.

14. The method of claim 13, wherein determining whether the user of the hearing instrument perceived the sound comprises:
determining, by the at least one processor, based on the motion data, a degree of rotation of a head of the user;
determining, by the at least one processor, whether the degree of rotation satisfies a motion threshold; and
determining, by the at least one processor, that the user perceived the sound in response to determining the degree of rotation satisfies the motion threshold.

15. The method of claim 14, wherein determining the motion threshold is based on one or more characteristics of the sound.

16. The method of claim 13, further comprising:
receiving, by the at least one processor, sound data indicating a time at which the sound was detected by the hearing instrument,
wherein determining whether the user perceived the sound is further based on the time at which the sound was detected by the hearing instrument.

17. The method of claim 16, wherein determining whether the user perceived the sound comprises:
determining, by the at least one processor, based on the motion data, a time at which the user turned a head of the user;
determining, by the at least one processor, an amount of elapsed time between the time at which the user turned the head of the user and the time at which the sound was detected; and
determining, by the at least one processor, that the user perceived the sound in response to determining the amount of elapsed time does not satisfy a time threshold.

18. The method of claim 13, wherein determining whether the user of the hearing instrument perceived the sound is based at least in part on a direction the user turned a head of the user.

19. The method of claim 18, further comprising:
determining, by the at least one processor, based on one or more characteristics of the sound, a direction of an audio source that generated the sound; and
determining, by the at least one processor, that the user perceived the sound in response to determining that the direction the user turned the head is aligned with the direction of the audio source.

20. A non-transitory computer-readable storage medium comprising instructions that, when executed by at least one processor of a computing device, cause the at least one processor to:
receive motion data indicative of motion of a hearing instrument;
determine, based on the motion data, whether a user of the hearing instrument perceived a sound, wherein execution of the instructions that cause the at least one processor to determine whether the user of the hearing instrument perceived the sound comprise instructions that, when executed by the at least one processor, cause the at least one processor to:
determine, based on the motion data, a degree of rotation of a head of the user;
determine a motion threshold based on at least one of age of the user, attention span of the user, cognition of the user, or motor function of the user;
determine whether the degree of rotation satisfies the motion threshold; and
determine the user perceived the sound in response to determining the degree of rotation satisfies the motion threshold; and
responsive to determining whether the user perceived the sound, output data indicating whether the user perceived the sound.

21. (canceled)
